SEQUENCE LISTING (8 sequences)

SEQ ID NO: 1 - DNA, 20 bp, Artificial
  primer for the Ltp1 promoter
cctgagcgggagatacaatc

SEQ ID NO: 2 - DNA, 20 bp, Artificial
  primer for the Ltp1 promoter
gatgagcaactcgtggaggt

SEQ ID NO: 3 - DNA, 20 bp, Artificial
  primer for the C1 gene
cgaaggaaggcgttaagaga

SEQ ID NO: 4 - DNA, 18 bp, Artificial
  primer for the C1 gene
ggcacttccctccatttg

SEQ ID NO: 5 - DNA, 21 bp, Artificial
  primer for the Bperu gene
ttctggtccatttcaagcact

SEQ ID NO: 6 - DNA, 19 bp, Artificial
  primer for the Bperu gene
ccacggagtggagatctta

SEQ ID NO: 7 - DNA, 18 bp, Artificial
  primer for the nos terminator
gaatcctgttgccggtct

SEQ ID NO: 8 - DNA, 22 bp, Artificial
  primer for the nos terminator
aattgcgggactctaatcataa

(19) United States
(12) Patent Application Publication
(10) Pub. No.: US 2008/0134360 A1
(43) Pub. Date: Jun. 5, 2008

(54) IDENTITY PRESERVATION SYSTEM FOR TRANSGENIC WHEAT AND TRITICALE

(75) Inventors: Robert J. GRAF, Lethbridge (CA); Francois EUDES, Lethbridge (CA); Andre LAROCHE, Lethbridge (CA); Denis A. GAUDET, Lethbridge (CA); Ketan M. DOSHI, Lethbridge (CA)

Correspondence Address: CAESAR, RIVISE, BERNSTEIN,;COHEN & POKOTILOW, LTD., 11TH FLOOR, SEVEN PENN CENTER, 1635 MARKET STREET, PHILADELPHIA, PA, 19103-2212, US

(73) Assignee: Her Majesty the Queen in Right of Canada, as Rep. by the Minister of Agriculture and Agri-Food, Lethbridge (CA)

(21) Appl. No.: 11/424,091

(22) Filed: Jun. 14, 2006

(51) Int. Cl. A01H 5/00

(52) U.S. Cl. 800/284; 800/320.3

(57) ABSTRACT

The growing controversy over GM/novel-trait crops and food products that possess novel industrial or nutritional traits, has resulted in an increased need for technologies that allow the rapid and reliable identification of GM/novel-trait crops so that they can be identified, and segregated if necessary, at the various stages of production and processing. A visual seed identity system is described that permits the identification and tracing of each individual seed through the upstream value chain, from field increase to the processing plant. These traits are genetically controlled, transmitted to progeny by either pollen or ovule and expressed in developing seed, showing a xenia effect. That novelty of this proposed IP system will facilitate early mitigation measures if out-crossing or admixtures occur and exceed a threshold limit set by regulatory agencies.

### FIELD OF INVENTION

The present invention relates to a process of identity preservation for cereal crops and the grain they produce. In particular, the invention relates to a method of identifying and tracing seeds, or fractions thereof, admixed with seeds from a different source using a seed colour marker that is dominant and is visually apparent in the hybrid seed. In one example the invention relates traceability of genetically modified (GM) crops or crops that express novel-traits (GM/novel-trait) and their genetic characteristics in selfing, out-crossing and admixture situations with traditional, non-GM/novel-trait crops

### BACKGROUND OF THE INVENTION

There is a spectrum of alternative procurement strategies for raw grains that can be adopted. Ultimately, buyers determine the elements of their procurement strategy. These can range from spot transactions, based simply on grade and non-grade factors, to full adoption of a separate, distinct system of grain production and handling (Wilson et al., 2003). Numerous terminologies exist to describe product differentiation systems. The leading terms are identity preservation (IP), segregation, and traceability. Frequently, the terminology surrounding this distinct system is used interchangeably (Smyth and Phillips, 2003). In the present invention, we will only focus on identity preservation and adopt definition by Sundstrom et al. (2002) “IP refers to a system of production, handling, and marketing practices that maintains the integrity and purity of agricultural commodities”. In general IP is the process of maintaining the identity of different crops. It involves separate production, storage, handling, documentation and testing of the crop's identity.

An IP system must be designed to provide assurances that the desired qualities or traits are present (or absent) in a product from the seed source, through all steps of production and delivery, to the end user. IP requires a set of procedures to ensure traceability and is usually communicated to the consumer by a label. Thus, IP results in additional costs for supply (European Commission, 2000). To reiterate, IP does not necessarily involve GM or novel trait-crops but IP may be desired when the end-user requires assurances that specific characteristics are present in the grain (Massey, 2002).

A common example of an IP grown crop under contract is the production of pedigreed seed. Contamination by foreign pollen or other seed varieties must be minimized and inspections take place to verify purity. In the case of wheat, the premium for certified seed is about 15-20% of the price of a normal wheat crop. This premium should cover the extra work involved for IP (European Commission, 2000). Other examples of IP systems already in place include high erucic acid rapeseed grown for industrial use, waxy corn for starch production, and flint corn for breakfast cereals in Europe. In the US, approximately 100,000 tons of soybeans are identity preserved, compared to 75 M tons harvested under the commodity system (European Commission, 2000). Variety choice through IP is seen as contributing more than any other factor to improve the market value of grains (Clarkson, 1999). A comparison of recent US prices shows that the premium paid for certain quality traits is much higher that the current premium for conventional crops. For example, in the health food sector, the price for IP grains and soybeans is about 200-300% of the commodity price (Cargill, 1999).

Identity Preservation necessitates implementation of a rigorous system of standards, records, and auditing that must be in place throughout the entire crop production, harvesting, handling and marketing process. Pedigreed seed production (seed certification) is an example of a successful IP program. Seed certification programs have been highly successful in maintaining the integrity of crop varieties and in providing farmers with seed of known pedigree with high purity and quality. As IP programs are developed for GM/novel-trait crops or agricultural commodities other than seed for propagation, they often follow principles similar to those used in seed certification. Thus, in describing the components of IP programs, seed certification is often used as the model. As the seed and food industries developed, the purity and quality expectations of buyers and processors increased and standards were established. Seed certification programs such as those used by Seed Certification Agencies, play a major role in maintaining seed purity standards at levels established by the industry for national and international trade. Similarly, commodity traders, marketing organizations, and food processors have established purity and quality tolerances for specific end product uses. As crop and production systems have diversified to meet market demands, the need for identity preservation has increased (Sundstrom et al., 2002). Further, the IP of GM/novel-trait crops is also essential to maintain the integrity of non-GM crops by preventing contamination of traditional crops by GM/novel trait crops and visa versa.

The growing controversy over GM/novel-trait crops and food products that possess novel industrial or nutritional traits, has resulted in an increased need for technologies that allow the rapid and reliable identification of GM/novel-trait crops so that they can be identified at the various stages of production and processing. This would also permit the regulatory agencies and the general public to make informed decisions regarding the utilization and consumption of such products. Such GM/novel-trait crops or their products may not be allowed to mix with traditional non-GM crops since, in some instances, the GM/novel-trait product will not be suitable for ingestion by humans or the livestock, or for inclusion in other household products (Caswell, 2000).

Growing genetically modified (GM/novel-trait) crops side by side with traditional crops creates a serious regulatory dilemma. How does one prevent cross contamination of the traditional crops, such as wheat and barley, with new GM/novel-trait crops? The situation is aggravated by the fact that future GM/novel-trait crops will include those for bio-industrial purposes, designed specifically to produce industrial products that are not intended for animal or human consumption (e.g.: bioplastics, adhesives, and specific starches). The introduction of bio-industrial triticale or wheat production, which would co-exist with traditional farming systems will necessitate development of technologies designed specifically to prevent cross-contamination of these two production streams by accidental admixture. In the event of an accidental admixture of GM/novel-trait and traditional crops, strategies must be in place to permit the rapid, effective detection of cross-contamination so that timely remedial action can be taken.

Therefore, we have developed an Identity Preservation (IP) system for GM/novel-trait crops that will permit individuals involved in the industry (including but not limited to the farmer, seed agent, elevator operator, industrial processors and regulatory agencies) to rapidly, efficiently, and effectively distinguish traditional crops from those GM/novel-trait crops and keep these two crop streams separate. This invention will also permit the determination of the extent of genetic out-crossing and admixture between traditional crops and GM/novel-trait crops, thereby permitting both traditional and GM/novel-trait streams to coexist in separate streams in the same production system.

The introduction of triticale as a specific bio-industrial crop will be an important first step in preventing cross-contamination of the traditional and bio-industrial crops because: 1) the triticale plant is easily distinguished from other traditional cereal crops such as wheat, barley, oat, and corn based on spike morphology; and 2) the triticale seed is easily distinguished from other traditional cereal crops such as wheat, barley, oat, and corn based on seed morphology. Triticale is a synthetic crop, created artificially through the laboratory hybridization of wheat and rye. An advantage of employing triticale as a crop for bio-industrial production is that triticale will very rarely out-cross outside the laboratory to its wheat and rye progenitors, and will never out-cross to any of the related cereals or grasses, thereby ensuring genetic isolation of recombinant DNA introduced into triticale. However, triticale will hybridize with itself, thereby creating potential problems when bio-industrial triticale is grown in proximity with triticale destined for traditional food or feed consumption.

Physical admixtures of other cereals with triticale, or visa versa, can be detected visually in very small seed lots because the seeds are morphologically distinct. However, in larger seed samples such as those likely to be encountered on-farm or at a grain elevator, visual detection of a few or even a single triticale seed among millions of wheat or barley seeds would be nearly impossible without some type of electronic or DNA-based system. DNA-based tests could be developed with existing technologies and would be sufficiently sensitive to identify a small amount of triticale seed contamination in a wheat or barley grain sample, but these are not yet available. Additionally, it would only be practical to conduct these tests on a small number of samples since it would be expensive, time-consuming, and require specialized equipment. Further, sampling for DNA-based tests are destructive, thus preventing additional analyses using other methods. In practice, when the presence of transgenic seed is suspected, DNA-based tests are used on a bulk sample and, as not all seeds can be tested, sampling error becomes an issue. These methods present an efficiency limit at about the threshold level allowed in EU (0.9%) or Japan.

Electronic detection systems for such an admixture are currently not available but could be developed with existing technologies. Electronic detection systems for detecting other seed abnormalities such as disease infection, mechanical damage, and physiological disorders, could be modified to detect triticale but these systems are currently expensive and not universally available. Genetic contamination could not be detected with an electronic detection system. In every case these methods require specialized equipment and are generally performed in a laboratory setting.

Thus, there is a need for a simple method of identifying and tracing seeds, or fractions thereof, admixed with seeds from a different source. The present invention relies on seed colour produced by anthocyanin genes, as a means to develop this type of segregation, traceability and IP method. Identification of seeds or plants using seed colour as a marker has been previously described in, for example: CA 2,436,203; CA 2,191,441 or CA 2,259,949. The present invention, however, differs from the prior art in that the anthocyanin genes that show an immediate effect (xenia) have been used; that is, the trait is dominant and is visually apparent in the hybrid seed.

### SUMMARY OF THE INVENTION

The present invention is for the identity preservation of cereal grains. In particular, the invention relates to the traceability of particular genetic characteristics of wheat and triticale in selfing, out-crossing and admixture situations.

The present invention provides a method of identifying and tracing seeds, or fractions thereof, admixed with seeds from a different source using a seed colour marker that is dominant and is visually apparent in the hybrid seed.

In a further embodiment of the present invention, a method for identifying and tracing seeds, or fractions thereof, admixed with seeds from a different source using a seed colour marker that is dominant and is visually apparent in the hybrid seed, wherein the method includes introducing a seed colour marker from wheat into triticale, is described.

In a further embodiment of the present invention, a method of identifying and tracing seeds, or fractions thereof, admixed with seeds from a different source using a seed colour marker that is dominant and visually apparent in the hybrid seed, wherein the method includes introducing into a wheat or triticale cultivar Bperu and C1 regulatory genes under the control of an embryo-specific promoter or a endosperm-specific promoter whereby producing wheat or triticale plants with a distinct tissue colour, is described.

In a further embodiment of the present invention, a method is provided for identifying and tracing seeds, or fractions thereof, admixed with seeds from a different source using a seed colour marker that is dominant and is visually apparent in the hybrid seed, wherein the method includes producing a wheat cultivar with a black seed colour by crossing a wheat with a blue seed colour with a wheat with a purple seed colour.

In yet a further embodiment there is provided a triticale plant displaying a seed colour selected from the group consisting of: blue, purple, red, grey, brown and black, provided by crossing a triticale plant with a wheat plant having a blue or purple seed colour.

In yet a still further embodiment there is provided a wheat or triticale plant transformed with Bperu and C1 regulatory genes under the control of a tissue specific promoter selected from an embryo-specific promoter and an endosperm-specific promoter whereby producing wheat or titicale plants with a distinct tissue colour.

This summary of the invention does not necessarily describe all features of the invention.

### BRIEF DESCRIPTION OF THE DRAWINGS

These and other features of the invention will become more apparent from the following description in which reference is made to the appended drawings wherein:

FIG. 1 shows a comparison of anthocyanin expression in transgenic and non-transgenic embryo of mature seeds in accordance with an embodiment of the present invention; a=wheat; b=triticale; arrow head indicates the expression of anthocyanin in the seeds (Bar=1 mm).

### DETAILED DESCRIPTION

The present invention relates to the identity preservation of cereal grains. In particular, the invention relates to the traceability of particular wheat and triticale genetic characteristics in selfing, out-crossing and admixture situations.

Red seed colour in wheat and triticale is produced by three dominant genes controlling the amount of phenolic compounds in the bran layers. These genes are inherited in the normal Mendelian fashion according to classical genetic theory. When all three loci express the recessive form of the gene, the seed is white. In triticale, the normal seed coat colour is red/amber, while the embryo is colorless (white). Anthocyanin pigments can also be expressed in the seed coat, to produce colours such as purple (seed coat), blue (aleurone layer), and black (superposition in both tissues). These colours occur in the genetic backgrounds of the progenitors of triticale (wheat and rye), but do not currently exist in triticale cultivars. The embryos of all cereals are always white.

Xenia is defined as a seed characteristic in which the hybrid nature of the seed is recognizable by particular attributes (colour, shape, size, etc.) owing to direct influences exerted by the phenotype of the pollen on the embryo or on the maternal tissue (endosperm) of the fruit (Focke, 1881). Blue seed coat colour in hexaploid wheat is controlled by a single dominant gene that exhibits xenia. The blue seed coat colour characteristic transferred from wheat to triticale is also a single dominant gene, which also exhibits xenia.

The seed or grain of cereals is comprised of the following tissue types: endosperm tissue, bran tissue and germ tissue. Endosperm Tissue is tissues comprising the vast majority of the cereal grain that contain the endosperm, cells filled with starch granules in a protein matrix, and cellulose which forms the cell walls. Bran tissues are comprised of the aleurone cell layer, (considered as part of the endosperm but separated with the bran), nucellar tissue, seed coat (testa), tube cells, cross cells, hyperdermis, and epidermis. Germ tissue includes the scutellum, sheath of shoot (coleoptile) rudimentary shoot, rudimentary primary root, root sheath, and root cap.

In the context of the present invention, a hybrid represents the result of a cross between two plants belonging to the same species (e.g. triticale) or between two plants belonging to different species (e.g. wheat crossed with rye).

A stable population of blue triticale (or wheat) exhibits xenia in the following manner. If a viable pollen grain from a blue triticale plant pollinates a conventional red triticale, the resulting seed will be blue. This phenotype is the result of the heterozygous nature of the seed in which the blue seed coat colour is dominant. If this hybrid blue seed is germinated and grown to maturity, each seed produced on the F₁ plant will reveal its genotype, in this case the expected ratio of blue to red seeds is 3:1, the normal Mendelian ratio for a single dominant gene at the F₂ generation.

The reverse situation, in which a floret within a stable population of blue triticale (or wheat) is pollinated by conventional red triticale, does not allow detection of the hybrid nature of the resulting seed. This is again due to the dominant nature of the blue seed coat trait. Detection of the cross pollination would require growth of the harvested seed. Any F₁ seeds would produce F₁ plants in which the seed produced within the spike would segregate for blue and red seed coat colour at the expected Mendelian ration of 3:1.

According to the present invention three strategies have been developed:

(1) Using standard interspecific hybridization protocols, the blue seed colour from wheat was introgressed into AC Alta, a Canadian triticale cultivar developed by Agriculture and Agri-Food Canada. This blue aleurone layer trait is monogenic, dominant and exhibits xenia.

(2) Using genetic engineering, the Bperu and C1 regulatory genes where introduced in wheat cv Superb and triticale cv AC Certa under the barley Ltp1 embryo specific promoter. Transgenic purple embryo wheat and triticale plants were regenerated. This purple embryo trait is monogenic, dominant and exhibits xenia.

(3) Wheat with black seed was produced in winter and spring lines from crosses using parents exhibiting the blue aleurone trait (1 gene) and purple seed coat colour (2 genes). This black seed trait is polygenic, not commercially grown. When out-crossing occurs, in which pollen from the black wheat pollinates a traditional red or white wheat, the blue aleurone trait is visible in the F₁ (hybrid) seeds.

Some colours such as blue aleurone anthocyanin, and purple embryo are directly expressed in the seeds that arise from a cross (xenia) and could be used to detect genetic contamination through out-crossing by errant GM pollen, because it would be immediately expressed in the developing seed. Other colour traits such as purple seed coat are expressed in subsequent segregating generations and would be suitable only for detecting admixtures when genetically fixed in a stable line or variety, but not for immediate detection of genetic contamination.

Thus, according to the present invention, genetically controlled colour markers are utilized to rapidly and reliably identify transgenic seed admixtures and genetic contamination from transgenic triticale and wheat. For this purpose, a built-in visual marker(s) system has been incorporated into every transgenic triticale seed. Empirically, we have determined that a single blue, purple, or black wheat or triticale seed, or a wheat or triticale seed with a purple embryo, can be easily detected visually in a kilogram (approximately 15,000 seeds) a red or white wheat, barley, or other cereal grains.

The grey and black seeded triticale and wheat, which are the result of genotypes possessing the gene for blue seed coat colour as well as one or two genes for purple seed coat colour, also exhibit xenia but in a slightly different manner. The examples of the manner of detection of out-crossing between blue and red triticale apply; however, in the case where a pollen grain results in the successful fertilization of a triticale or wheat of traditional red or white seed colour, the resulting seed will be blue. The reciprocal pollination would require one generation of growth in order to detect the out-cross, in which the F₁ plants would produce seeds segregating for seed colour

Both of the examples in which out-crossing has occurred demonstrate a distinct advantage of the xenia effect to detect and trace transgenic plants. For example, in transgenic canola, the detection of cross pollination requires one or two generations of plant growth, depending on the direction of out-crossing that is to be detected. Assuming that the herbicide resistance trait is dominant, the detection of pollen flow from a herbicide resistant plant to a non-herbicide resistant plant would require harvest of the F₁ seed, planting of a large population of these seeds, and detection of the herbicide resistant trait by application of the herbicide in question. For the opposite situation of pollen flow from a conventional non-herbicide resistant canola to a herbicide resistant canola, two generations of propagation is required (the F₁ seed and resulting F₂ seed) with herbicide application in the F₂ generation for detection of the plants resulting from the out-cross. This would be the expected result in CA 2,436,203.

This invention thus allows the traceability of each individual GM/novel-trait seed through the upstream value chain, from the field to the processing plant. These traits are genetically controlled, transmitted to the progeny genetically and expressed in the developing seed, showing the xenia effect. The novelty of this proposed IP system is that it facilitates early and appropriate mitigation measures if out-crossing or admixture occurs and exceeds a threshold limit set by regulatory agencies.

Novel traits are defined as any traits that do not currently exist in the current, traditional classes of crops grown in Canada, North America, or worldwide, that affect the appearance or quality of the plant or grain, and that, by their presence even in small quantities, would reduce the value of the traditional crop. Novel traits are genetically controlled and can be generated by mutagenesis or can originate from wild or related species and transferred by interspecific hybridization. Novel traits can be inserted into any crop species by conventional breeding techniques or by genetic modification. Genetic modification can be defined in various ways, it is used herein to indicate the use of recombinant DNA technologies to introduce genes into plants (Suslow et al., 2002). The expression of those genes in plants results in specific production or quality traits.

To prevent or minimize problems associated with out-crossing of GM bio-industrial triticale to non-bio-industrial triticale or other cereals, or the accidental admixture of bio-industrial triticale with other triticale or other cereals, we are introducing seed colour marker(s) that will permit easy visual detection of contamination. Similar industrial applications could be considered in wheat as well, or other input and output traits as seen with FHB resistant wheat in USA. Traceability and the means to reduce the risk of harm to non-GM/novel-trait crops from the field to the processing plant can be addressed using the described genetic IP system incorporated into each seed and expressing the xenia effect (the blue aleurone trait is visible in the seed produced by the parental plant).

Under normal growing conditions, cross-pollination resulting in the hybridization between a GM/novel-trait plant and non-GM plant of the same crop species can occur. The level of cross pollination has the potential to be high (approaching 100%) in “cross-pollinated” species (e.g. rapeseed, canola, rye) and very low (in a range of 0% to 5%) in “self-pollinated” species (wheat, triticale, barley, flax)(Gustafson et al., 2005; Hanson et al., 2005). Inadvertent admixtures or misidentification of grain have occurred during handling and transport and are likely to occur in the future at a low frequency. In order to preserve any product being produced in a GM/novel-trait crop and its intrinsic market value, the ability to identify and quantify the presence of non-transgenic contamination is of fundamental importance to the industry so as to facilitate the assessment of the true value of seed lots (including price and discounted value due to the presence of GM seeds). (Segarra and Rawson 2001). For example, the commercial value of a specialized starch-modified seed lot could be nullified if mixed up with regular seeds.

Furthermore, in order to preserve the commercial value of traditional crops, the ability to identify and quantify the presence of GM/novel-trait seed contamination is of fundamental importance to the seed industry so as to be able to better assess the real value of seed lots, including the price and discounted value due to the presence of GM/novel-trait seeds. Commercial value of a traditional crop could be readily nullified if confused with the GM/novel-trait crop. Furthermore, transportation companies, seed brokers, grain handlers and seed producers could be hit with heavy fines that could compromise their financial stability and sustainability.

Cereals, in addition to their direct use as animal feeds, have innate value based on their seed composition. Milling is the first processing step in which the grain constituents are separated, and concentrated to add value to the raw grain product. In brief, milling starts with chemical analysis that classifies the grain and its constituents. The grain is then cleaned, tempered, and finally milled to yield its inner constituents. Following this, the constituents are separated into various fractions, including bran, shorts, flour, and germ.

Genetic modification using DNA technologies offers the promise of adding novel traits into the raw grain or modifying the concentration and proportions of the existing constituents. Production of the new traits can be directed to specific tissues including the seed coat, aleurone layer, endosperm, and the germ, by using specific promoters. This ultimately permits the fractionation of novel products using standard or available milling procedures and equipment.

Bioindustrial production will result in the generation of novel products in the plant and seed. Certain novel products may pose harm to human or animal health if consumed and thus, processing of these products would require separate facilities. However, the majority of novel products are not likely to have adverse affects on human health if consumed, and some novel products are expected to have beneficial health effects (e.g.: digestion-resistant starches, oligofructans, vitamins, etc.). Grains that produce novel products that do not pose any risk to human health may be batch-milled in existing commercial mills using existing protocols and equipment.

The biochemical basis of the colour markers described elsewhere in this patent application is related to the accumulation of different quantities and types anthocyanin, and the expression of anthocyanin is via DNA-based gene action. For example, expression of a red anthocyanin can produce a colour ranging from brown to purple depending on the amount produced. Additionally, there are other anthocyanins known to impart blue colours. By combining various levels of different markers, a wide range of colours are possible in the grain. In general, the naturally occurring seed colours existing in wheat and other grains are expressed in the seed coat and aleurone layer, which form part of the bran fraction upon milling. Thus, by expressing a novel trait in the embryo or the endosperm tissues, it will be possible to separate the colour marker from the fraction containing the novel product. However, anthocyanin genes placed under embryo-specific or endosperm-specific promoters will also express colour in the embryo or endosperm, respectively. Thus, by employing the same promoter to express both the anthocyanin gene and the novel trait, it will be possible to link the colour marker with fractions containing the novel trait. This would be very useful in a milling situation, since fractions containing novel traits would be visually distinguishable from those fractions that do not contain the novel traits, permitting easy segregation and identification of fractions containing novel products within a conventional milling facility. By expressing different anthocyanins or different levels of the same anthocyanin in different seed layers, it will be possible to express more than one novel trait in different regions of the seed, and, by co-expressing a different anthocyanin gene or different levels of the same anthocyanin gene with different novel traits, it will be possible to link different colours to different novel products produced within the same seed, for example the bran and the germ. This aspect will be very useful in a conventional mill where several food-based products are produced simultaneously and could be visually distinguished based on the colour of the fraction. This feature would also be useful in non-food industrial mills where several industrial products are extracted from single crop, or from different crops, using a common extraction process. This aspect of the invention can also be used in a air-classification milling process that separates particles according to density.

A number of tissue specific plant promoters are known and can be used in the present invention. The following list is not exhaustive, the present invention can utilize other tissue specific plant promoters. Two barley aleurone-specific promoters from genes encoding lipid transfer protein (Ltp1) and chitinase (Chi26), respectively, were transcriptionally fused to a B-glucuronidase gene (GUS). Tissue-specific expression of these GUS chimeric constructs was examined in transgenic rice. The Ltp1 promoter was not expressed in the aleurone layers but it was expressed in embryo tissue (Hwang et al., 2001).

In transgenic tobacco, the Itr1 promoter drives expression of the GUS reporter gene, not only in developing endosperm but also in the embryo, cotyledons and the meristematic intercotyledonary zone of germinating seedlings (Diaz et al., 1995).

In bean, the expression of the B-phaseolin gene (phas), which encodes the major seed storage protein of bean, is confined to the cotyledons of developing embryos. Seeds from plants transformed with the proximal 227 bp of promoter showed embryo-specific GUS activity (van der Geest and & Hall, 1996).

Expression of the GS1-2 promoter/GUS heterologous gene resulted in the predicted tissue specific expression in the basal maternal seed tissue, including the surrounding pericarp. Gene expression within the pedicel parenchyma that subtends the basal endosperm transfer cells and embryo was particularly strong. In contrast, GUS staining was absent in the endosperm and embryo, as well as in leaves or roots (Muhitch et al., 2002).

Cotton α-globulin promoter was fused to GUS in the binary vector and its expression pattern was studied in cotton, Arabidopsis and tobacco. Histochemical analysis revealed that the promoter began to express during the torpedo stage of seed development in tobacco and Arabidopsis, and during the cotyledon expansion stage in cotton. The activity quickly increased until embryo maturation in all three species (Sunilkumar et al., 2002).

Analysis of transcriptional or translational fusion between the barley Lox1 5′ upstream sequence and the gusA reporter gene indicated that the 5′-untranslated leader sequence was involved in embryo specific expression (Rouster et al., 1998).

To define the regions of the maize alcohol dehydrogenase 1 (Adh1) promoter that confers tissue-specific expression, a series of 5′ promoter deletions and substitution mutations were linked to the GUS reporter gene and introduced into rice plants. A region between −140 and −99 not only conferred anaerobically inducible expression in the roots of transgenic plants but was also required for expression in the root cap, embryo and in endosperm under aerobic conditions (Kyozuka et al., 1994).

The Dhn12 (dehydrin) gene is located on chromosome 6H of barley, and shows a different expression pattern from all other Dhn genes identified previously. RT-PCR results show that Dhn12 is embryo specific (Choi et al., 2000).

A wheat puroindoline gene promoter was detected only in seeds and within seeds. Histological localization showed GUS activity as being restricted to the endosperm, aleurone cells and pericarp cell layers; no GUS activity was detected in the embryonic axis (Digeon et al., 1999).

A promoter fusion (Sh35) combining upstream regulatory regions from the maize Sh1 promoter with a truncated 35S promoter was directing 25-fold higher GUS expression in tomato seeds (D'Aoust et al., 1999).

The promoter of the pea lectin (psl) gene, encoding an abundant seed protein, was fused with the GUS gene and transformed in tobacco. Expression increased during the mid-maturation stage of seed development and was observed in both the endosperm and the embryo; expression decreased dramatically within a few days after germination (de Pater et al., 1993).

The abscisic acid-responsive gene rab17 and rab28 are induced during maize embryo maturation and in vegetative tissues under water stress conditions (Pla et al., 1993; Vilardell et al., 1994).

The promoter region (−309 to +44) of the Brassica napus storage protein gene napA was studied in transgenic tobacco by successive 5′ as well as internal deletions fused to the GUS reporter gene. The expression in the two main tissues of the seed, the endosperm and the embryo, was shown to be differentially regulated (Ellerströ m et al., 1996).

The USP promoter from Vicia faba becomes active in transgenic tobacco seeds in both the embryo and the endosperm, whereas its activity in Arabidopsis is detectable only in the embryo (Baumlein et al., 1991).

Feed represents a major cost in livestock production, with raw grains forming a substantial component of animal diets. Different livestock types, and even different breeds of livestock, vary in their ability to utilize feed efficiently. DNA technologies now permit expression of novel traits that can positively impact digestibility, feed-efficiency and nutritive properties of raw grains as animal feed. Depending on the animal species, the breed, and the growth stage, the feed ration will change during the various stages of growth and finishing. As feed technology advances, animal diets will become increasingly complex. Using specific seed colours to designate different nutritional attributes inserted into course grains by recombinant DNA technology would facilitate segregation of on-farm storage and improve livestock production efficiency. As increasingly improved technologies become available to the feed industry, the need for a rapid, efficient and effective visual identification system for grains will increase.

The present invention will be further illustrated in the following examples.

### EXAMPLE 1

### Transfer of Unique Seed Coat Colours from Wheat to Triticale

Genetic transfer of blue and purple seed coat colour from wheat to triticale was initiated through the use of standard crossing procedures for triticale (Larter and Gustafson, 1980) and wheat (Allan, 1980).

Both the triticale and wheat plants were grown under greenhouse conditions, with two seeds planted per 1 gallon pot containing Cornell artificial soil mixture. Lighting and temperatures conditions were a 16 photoperiod, with a 21/15° C. diurnal temperature. The triticale was common seed of the cultivar AC Alta. The blue and purple wheat were experimental lines designated “Purendo38” and “Purple Bulk”. The blue seeded wheat, “Purendo38” was obtained from Dr. Pierre Hucl of the University of Saskatchewan, College of Agriculture. Department of Plant Sciences. The purple seeded wheat, designated “Purple Bulk”, was a composite of several early generation plants exhibiting purple seed coat colour, derived from a back-crossing program to experimental spring wheat lines in which the source of the purple seed trait was the New Zealand cultivar “Konini”. Several pots of the wheat parents were seeded at one week intervals for five to six weeks following the planting of the triticale to ensure that cross pollination of wheat and triticale could be achieved. The plants were fertilized with 20-20-20 liquid plant fertilizer approximately every two weeks.

The first step in the triticale X common wheat hybridization was the removal of all of the anthers from selected spikes of the triticale plant, which served as the female parent. This step is referred to as emasculation. Spikes of the female parent were selected for emasculation based on morphological development, which was approximately one day after the spike emerged from the boot. Emasculation at this stage ensured that the anthers were green and that self pollination had not occurred.

To emasculate the triticale spike, all of awns were cut off using scissors. The upper and lower one or two spikelets were then removed from the spike using forceps. In addition, all but the primary and secondary florets of each spikelet were removed by pulling them out with forceps. Following removal of these florets, the three anthers from each of the primary and secondary florets were removed using forceps, using care to ensure that the stigmas were not damaged. The spike was then covered with a glycine bag and secured with a paper clip to ensure that accidental cross pollination could not occur. The date of emasculation was written at the top of the bag for visibility.

Following emasculation, the triticale florets were pollinated with the wheat pollen after four to five days, ensuring that the stigmas were well developed and feathery. At the time of pollination, the glycine bag was removed and the top one-third of florets of the triticale female were clipped back to facilitate access to the stigmas. Spikes of the wheat parent with suitable pollen were identified by the extrusion of fresh anthers. Mature pollen was obtained from florets in close proximity to those that had been newly extruded.

Wheat anthers dehiscing fresh pollen were carefully brushed against the receptive stigmas of the emasculated triticale florets, using forceps to hold the anther. Usually, one anther was used to pollinate two florets. Following pollination of the entire spike, a small tag was affixed to the culm to indicate the female and male parent plants utilized and the date of pollination. The glycine bag was then put back over the spike.

Approximately 2000 pollinations were attempted, with the development of 550 small, variably shriveled, triticale x wheat hybrid seeds. This rate of approximately 27.5% successful hybridization in hexaploid triticale x common wheat is similar to that reported in the literature. The crossing success was similar for the blue and purple wheat parents.

Following the successful harvest of the mature triticale x wheat hybrid seeds, the first backcross to triticale was initiated. Several of the plumpest hybrid seeds were planted from each of the crosses (triticale x blue wheat, triticale x purple wheat), as well as AC Alta triticale. Seeding was done at several dates to ensure that plant development was synchronized so that cross pollination could be achieved. Growth conditions were similar to those used for the initial cross described above. For this cross, the triticale x wheat hybrid was used as the female parent. For the transfer of the blue seed coat colour, the hybrid seeds (BC₂F₁) that appeared blue were planted. There was no indication of purple colour in the triticale x purple wheat hybrid.

This process was repeated an additional three times for the transfer of blue seed coat colour to triticale. Following the fifth cross (fourth backcross=BC₄), the blue coloured hybrid seeds were harvested and planted under the greenhouse conditions previously described. At maturity, these BC₄F₁ plants were harvested individually. Plants that were excessively tall were discarded prior to harvest. Seed from each harvested plant was examined for kernel plumpness, kernel size, and intensity of blue colouration. Those plants that produced large, plump kernels of intense blue colouration were identified for seeding the BC₄F₂ generation. Only blue seeds were planted. During growth and development, those plants that were excessively tall were discarded, as in the BC₄F₁ generation.

Plants were harvested individually at maturity, recording the original BC₄F₁ plant from which the seed was derived. Since the blue seed coat gene exhibits xenia, as was demonstrated by the expression of the blue seed colour in the hybrid seeds, the expected ratio of plants that produced completely blue seed compared to plants that produced both blue and red seed in the same spike was 1:2, since only blue seeds were planted. The expected ratio of blue to red seeds on those plants producing both colours was 3:1, which is the segregation ratio for the progeny of a heterozygote expressing a single gene trait.

Seed from plant families that conformed to the expected Mendelian segregation ratio and were completely blue were seeded as individual plant derived BC₄F₃ rows. Selection of rows continued based on standard agronomic criteria, such as plant vigour, height, straw strength, fertility and maturity. Individual heads were selected from the best rows, taking note which row each head was selected from. Each row was harvested individually at maturity, with the grain examined for blue colouration. Those rows that were not completely blue were discarded, as were the heads selected from those rows.

At this stage, the blue seed coat colour was considered to be fixed within triticale, and standard plant breeding techniques were utilized for development of a blue seeded triticale cultivar that was distinct, uniform and stable

For the transfer of the purple seed coat colour, the BC₁F₁ seeds were planted under greenhouse conditions and grown to maturity. Plump seeds that exhibited purple colouration (BC₁F₂) were planted and used as pollen donors for another backcross to AC Alta triticale. The resulting BC₂F₁ seeds were planted and used as donor plants for doubled haploid production using the protocol described by Eudes et al (2005). The doubled haploid (DH) plants that exhibited purple seed colour were considered to have this trait fixed.

The production of black triticale was accomplished by crossing the DH purple seeded plants with plants fixed for the blue seed trait. The resulting F₁ seeds were used as doubled haploid donor plants to produce completely homozygous lines exhibiting black seed coat colour, along with other lines exhibiting purple, blue and red seed coat colours as well as intermediates. Since it is believed that purple seed coat colour is expressed through the action of two genes, other seed coat colours were observed and are postulated to be a result of the following genetic combinations:

Red=no blue gene+no purple genes

Brown=1 purple gene

Purple=2 purple genes

Blue=blue gene

Grey=blue gene+1 purple gene

Black=blue gene+2 purple genes

### EXAMPLE 2

### Anthocyanin Expression in Transgenic Wheat and Triticale Embryos (Purple Visible Embryo Marker)

According to the present invention, we report the recovery of anthocyanin expressing embryo in mature seeds of wheat (Triticum aestivum L.) and triticale (XTritiosecale) lines, without using the selectable marker gene.

Plasmid pLtp1CB, which carries C1 (NCBI reference AF320613; AF320614) and Bperu (NCBI reference X57276; X70791) anthocyanin biosynthesis genes driven by barley Ltp1 embryo specific promoter (GenBank Accession No. X60292) was cloned in E. coli DH5α and purified using QIAGEN Plasmid Maxi kit (QIAGEN Inc., Mississauga, ON, Canada, Cat. No. 12162). The original constructs carrying C1 and Bperu genes is from Chawla et al. 1999. The resulting DNA pellet was resuspended in TE buffer (10 mM Tris-HCl, 0.1 mM EDTA, pH 8.0) and the gene cassette was excised by doubled digesting plasmid DNA with BamH1 and Sph1 restriction enzymes. Once the 5.45 kb DNA fragment was gel purified, DNA fragment concentration was quantified using spectrophotometry (BioMate 3, Thermo Electron Corp., Waltham, Mass.) at 260 and 280 nm and adjusted to 1.0 μg/μL. Cartridges were prepared for and according to the Helios™ Gene Gun System Instruction Manual (Bio-Rad Laboratories, Inc., Mississauga, ON, Canada, Cat. No. 165-2431 and 165-2432) by using 25 μg DNA, 25 mg of gold particles (1.0 μM) and 0.005 mg/ml PVP.

Wheat cv Superb and triticale cv AC Certa, were seeded in 6-in pots containing Cornell mix, and grown in growth chambers with a 16 h photoperiod (bottom/top of plants canopy, 270/330 μmol m⁻² s⁻¹) and 21/16° C. day/night temperature regimes. The tissue preparation, dissection and culture processes were performed according to Eudes et al. (2003). Twenty scutella were cultured on 20 mL DSEM medium in 15×100 mm Petri dish for 48 h. Then, scutella were grouped within a 2 cm diameter circle on the same DSEM plate and placed in the dark for 4 h at 25° C. prior to bombardment. Scutella were bombarded with the Ltp1CB fragment using the Helios gene gun, set at a delivery pressure of 140 psi. Negative control treatments consisted of five Petri dishes each containing 10 scutella per dish which were not bombarded. Sixteen hours following this treatment, scutella were regrouped on the same Petri dish. Plants were recovered and grown to maturity without using any selective pressure in the media.

Using PCR, putative T₀ transformants were pre-screened along with negative controls consisting of genomic DNA of wheat cv Superb and triticale cv AC Certa, and positive controls consisting of 10.5 ng genomic DNA spiked with 1.5 ng pLtp1CB plasmid DNA. 12 ng of DNA was heat denatured at 94° C. for 5 min prior to 45 PCR cycles: 45 s at 94° C., 30 s at 58° C. and 30 s at 72° C. PCR reactions were run for four sets of primers that were highly specific to different regions of the genetic cassette: Ltp1 promoter (5′CCTGAGCGGGAGATACAATC 3′ (SEQ ID NO.: 1) and 5′GATGAGCAACTCGTGGAGGT 3′(SEQ ID NO.: 2)); C1 gene (5′CGAAGGAAGGCGTTAAGAGA3′(SEQ ID NO.: 3) and 5′GGCACTTCCCTCCATTTG 3′(SEQ ID NO.: 4)); Bperu gene (5′TTCTGGTCCATT TCAAGCACT 3′(SEQ ID NO.: 5) and 5′CCACGGAGTGGAGATCTTA 3′(SEQ ID NO.: 6)) and nos terminator (5′GAATCCTGTTGC CGGTCT 3′(SEQ ID NO.: 7) and 5′ AATTG CGGGACTCTAATCA TAA 3′(SEQ ID NO.: 8)).

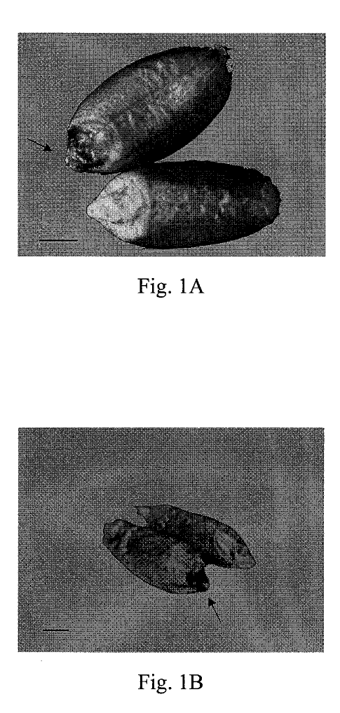

A total of 385 wheat and 432 triticale scutella were bombarded with Ltp1CB cassette. In vitro culture of isolated immature scutella of wheat cv Superb and triticale cv AC Certa produced direct somatic embryos within 8-9 days from initiation of cultures on DSEM. Scutella were viewed and evaluated under a stereo microscope for anthocyanin pigmentation two days post bombardment. No significant differences (P>0.05) were observed among the different batches of transformed scutella for cells expressing anthocyanin pigmentation and for color intensity, which demonstrated the reproducibility in DNA delivery to recipient tissues. The plantlets were regenerated following tissue culture on five consecutive steps and media, without using any selective pressure. The rooted plantlets were successfully established in greenhouse and grown into normal fertile plants. A total of 242 and 267 putatively transformed wheat and triticale lines were regenerated, respectively. Among them, only two wheat lines and a triticale line showed varying levels of anthocyanin pigmentation in embryo of mature seeds (FIG. 1); anthocyanin pigmentation was not observed in any other parts of the plants. Visual observations on more than 500 regenerants, grown to maturity under greenhouse conditions, showed that all plants were phenotypically normal and fully fertile.

Over all six wheat lines designated as W73, W90, W117, W194, W218 and W229 and 4 triticale lines designated as T165, T176, T213 and T237 were positive for Ltp1 promoter, C1 gene, Bperu gene and nos terminator. Overall, the transformation frequencies for Ltp1CB were 1.55% and 0.93% for cv Superb and cv AC Certa, respectively.

This result shows that anthocyanin expression in embryo (Visible Embryo Marker) is a dominant character and is also inherited to the next generation. Because, generally, T₀ plant was heterozygous and we got a transgenic lines in wheat and triticale with visible embryo marker in T₁ seeds of T₀ generation and further T₂ seeds of T₁ generation.

### EXAMPLE 3

### Crossing of 6× (hexaploid) and 8× (octaploid) Triticale for Seed Coat Color

Following crosses (F₁ and BC₁) were made:

Cross 1. 8× triticale 89TT108 (Female)×6× triticale blue TTT B-6-15 (Male)

Twenty eight seeds were obtained by this cross and all F₁ seeds were blue in color (observed xenia effect, as the male parent had blue seed color), which suggests that blue seed color is dominant over red seed color.

Cross 2. 8× triticale 90CT1ROW397 (Female)×6× triticale blue TTT B-6-15 (Male)

Thirty two seeds were obtained by this cross and all F₁ seeds were blue in color (observed xenia effect, as the male parent had blue seed color), which suggests that blue seed color is dominant over red seed color.

### EXAMPLE 4

### Development of Unique Seed Coat Colour Wheat

The majority of wheat available for commerce has either red or white seed coat colour. Red seed in wheat is conditioned through the action of three dominant genes which are additive in effect. Each of which resides on one of the wheat genomes (A, B, and D), each of which is homozygous for the recessive allele at each locus is white. As the dominant alleles are added, three gradations of red colour are recognized: light red, medium red and dark red.

Lines of common hexaploid wheat with both blue and purple seed coat colour are available in the public domain through various seed repositories. These colours are inherited independently of the red/white colour system described above. The blue seed coat colour exhibits xenia, the purple seed coat colour does not.

The simplest way to produce an array of unique seed coat colours in wheat is to cross-pollinate purple wheat with blue wheat using standard crossing practices; the direction of the cross is not important. The F₁ hybrid can then be used as a donor for doubled haploid production or grown to initiate a conventional breeding and selection program.

As described in the triticale example, an array of colours is possible using this technique in wheat.

Red=no blue gene+no purple genes

Brown=1 purple gene

Purple=2 purple genes

Blue=blue gene

Grey=blue gene+1 purple gene

Black=blue gene+2 purple genes

Black spring and winter wheat germplasm was created crossing P1281 (purple) and P1282 (black) parent seed, and fixing the trait following anther culture.

### EXAMPLE 5

### Preservation of Genetically Modified GM/Novel-Trait Crops

Under any growth conditions, hybridization between GM and non-GM plants of the same crop species can occur. The level of cross pollination has the potential to be high in cross-pollinated species (e.g. rapeseed, canola, rye) and very low if present in self-pollinated species (wheat, triticale, barley, flax). Inadvertent admixtures or misidentification of grain lots during handling and transport have occurred in the past and are likely to happen in the future. In order to preserve any product being produced in a GM/novel-trait crop, and its intrinsic market value, the ability to identify and quantify the presence of non-transgenic contamination is of fundamental importance (Segarra and Rawson 2001). For example, the commercial value of a specialized starch-modified seed lot could be readily nullified if mixed or confused with regular seeds. The impact of 1%, 3% and 5% cross contamination of triticale blue seeds, produced according to the present invention as in Example 1, by regular triticale seeds is obvious. The impact of 0.4, 1.2% and 4.8% cross contamination of wheat black seeds, produced according to the present invention as in Example 4, by regular wheat seeds is obvious. The level of contamination can be readily estimated by a visual assessment or could be automated using an advanced automated grain analysis technology such as the DuPont™ Acurum™ (http://www.acurum.com/home/) that uses digital imaging to objectively assess the fraction of non-transgenic seeds. The colour of the seed coat, color of the germ, or both can provide an easy and very effective tool to assess the presence of non-GM triticale and wheat seeds in GM/novel-trait triticale and wheat seeds. This type of system would be generally applicable to all crop species.

### EXAMPLE 6

### Protection of Non-GM/Novel-Trait Crops

Again, under any growth conditions, hybridization between GM and non-GM plants of the same crop species can occur. The level of cross pollination has the potential to be high in cross-pollinated species (e.g. rapeseed, canola, rye) and very low, if present, in self-pollinated crops (wheat, triticale, barley, flax). Inadvertent mixtures or misidentification of seed lots have occurred in the past (Segarra and Rawson 2001) and are likely to happen in the future during grain handling and transport. In order to preserve the commercial value of traditional crops, the ability to identify and quantify the presence of GM/novel-trait seed contaminations is of fundamental importance to the industry so as to facilitate the assessment of the true value of seed lots (including price and discounted value due to the presence of GM seeds). The commercial value of a traditional crop could be readily nullified if confused with the GM/novel-trait crop. Furthermore, transformation companies, seed brokers, grain handlers and seed producers could be hit with heavy fines that would compromise their financial stability and sustainability. The impact of 0.4%, 1% and 5% cross contamination of triticale blue seeds, produced according to the present invention as in Example 1, by regular triticale seeds is obvious. The impact of 0.4%, 1% and 5% cross contamination of wheat black seeds, produced according to the present invention as in Example 4, by regular wheat seeds is obvious. The level of contamination can be readily estimated by a visual assessment or could be automated using an advanced automated grain analysis technology such as the DuPont™ Acurum™ (http://www.acurum.com/home/) that uses digital imaging to objectively assess the fraction of non-transgenic seeds. The color of the seed coat and color of the germ or both provide an easy and very effective tool to assess the presence of GM triticale and wheat seeds in traditional crop and more generally, this concept would be applicable to all crop species

### REFERENCES

Allan R E. 1980. Wheat. Pp. 709-720 in: W. R. Fehr and H. H. Hadley, H. H. (eds.) Hybridization of crop plants. Amer. Soc. Agron., Madison, Wis., US
Baumlein H, Boerjan W, Nagy I, Bassuner R, Van Montagu M, Inze D, Wobus U. 1991. A novel seed protein gene from Vicia faba is developmentally regulated in transgenic tobacco and Arabidopsis plants. Mol. Gen. Genet. 225:459-467.
Cargill. 1999. Segregation or identity preservation of GM foods. Note to the House of Commons Agricultural Committee Inquiry into the Segregation of GM Foods.
Caswell J A. 2000. Labelling policy for GMOs: to each his own? AgBioForum 3:53-57
Chawla H S, Cass L A, Simmonds J A. 1999. Expression of anthocyanin pigmentation in wheat tissues transformed with anthocyanin regulatory genes. Current Sci, 76:1365-1670.
Choi D-W, Rodrigez E M, Close T J. 2000. Barley Cbf3 Gene identification, expression pattern, and map location Theor. Appl. Genet. 100:1274-1278.
Clarkson L. 1999. GMO concern spurs interest in IP. Agra Europe Conference paper, 2-3, London.
D'Aoust M A J, Nguyen-Quoc B J, Le V Q J, Yelle S J. 1999. Upstream regulatory regions from the maize Sh1 promoter confer tissue-specific expression of the beta-glucuronidase gene in tomato. Plant Cell Reports 18:803-808.
Diaz I, Royo J, O'Connor A, Carbonero P. 1995. The promoter of the gene Itr2 from barley confers a different tissue specificity in transgenic tobacco Mol. Gen. Genet. 248:592-598.
de Pater S, Pham K, Chua N H, Memelink J, Kijne J. 1993. A 22-bp fragment of the pea lectin promoter containing essential TGAC-like motifs confers seed-specific gene expression. The Plant Cell 5:877-886.
Digeon J F, Guiderdoni E, Alary R, Michaux-Ferrière N, Joudrier P, Gautier M F. 1999. Cloning of a wheat puroindoline gene promoter by IPCR and analysis of promoter regions required for tissue-specific expression in transgenic rice seeds. Plant Mol. Biol. 39:1101-1112.
Ellerström M, Stålberg K, Ezcurra I, Rask L. 1996. Functional dissection of a napin gene promoter: identification of promoter elements required for embryo and endosperm-specific transcription. Plant Mol. Biol. 32:1019-1027.
Eudes F, Amundsen E. 2005. Isolated microspore culture of Canadian 6× triticale cultivars. Plant Cell, Tissue and Organ Culture 82:233-241.
Eudes F, Acharya S, Laroche A, Selinger L B, Cheng K-J. 2003. A novel method to induce direct somatic embryogenesis, secondary embryogenesis and regeneration of fertile green cereal plants. Plant Cell Tissue and Organ Culture 73: 147-157.
European Commission. 2000. Economic impacts of genetically modified crops on the Agri-food sector. http://europs.eu.int/comm/agriculture/publi/gmo.pdf
Focke W O. 1881. Die pflanzenmischlinge, eine neue episode aus der befruchtungs-geschichte. Anat. Anz. 6: 266.
Gustafson D I, Horak M J, Rempel C B, Metz S G, Gigax D R, Hucl P. 2005. An empirical model for pollen-mediated gene flow in wheat. Crop Sci. 45: 286-1294.
Hanson B D, Mallory-Smith C A, Shafii B, Thill D C, Zemetra R S. 2005. Pollen-mediated gene flow from blue aleurone wheat to other wheat cultivars. Crop Sci. 45:1610-1617.
Hwang Y S, Nichol S, Nandi S, Jernstedt J A, Huang N. 2001. Aleurone- and embryo-specific expression of the β-glucuronidase gene controlled by the barley Chi26 and Ltp1 promoters in transgenic rice. Plant Cell Reports 7:647-654.
Kyozuka J, Olive M, Peacock W J, Dennis E S, Shimamoto K. 1994. Promoter elements required for developmental expression of the maize Adh1 gene in transgenic rice. The Plant Cell 6:799-810.
Larter E N, Gustafson J P. 1980. Pp. 681-694. In: W. R. Fehr and H. H. Hadley, H. H. (eds.). Hybridization of crop plants. Amer. Soc. Agron., Madison, Wis., USA.
Massey R E. 2002. Identity preserved crops. Ag Decision Maker, Iowa State University. http://www.extension.iastate.edu/agdm.
Muhitch M H, Liang H, Rastog R, Sollenberger K G. 2002. Isolation of a promoter sequence from the glutamine synthetase gene capable of conferring tissue-specific gene expression in transgenic maize. Plant Sci. 163:865-872.
Pla M D E, Vilardell J D E, Guiltinan M J D E, Marcotte W R D E, Niogret M F, Pagès M. 1993. The cis-regulatory element CCACGTGG is involved in ABA and water-stress responses of the maize gene rab28. Plant Mol. Biol. 21:259-266
Rouster J, van Mechelen J, Cameron-Mills V. 1998. The untranslated leader sequence of the barley lipoxygenase 1(Lox1) gene confers embryo-specific expression. The Plant J. 15:435-440.
Segarra A E, Rawson J M. 2001. StarLink™ Corn Controversy: Background. http://cnie.org/NLE/CRSreports/Agriculture/ag-101.cfm
Smyth S, Phillips P W B. 2003. Product differentiation alternatives: identity preservation, segregation, and traceability. AgBioForum 5:30-42.
Sundstrom F J, Williams J, Deynze A V, Bradford K J. 2002. Identity preservation of agricultural commodities. University of California Division of Agriculture and Natural Resources, Agricultural Biotechnology in California Series 8077.
Sunilkumar G A, Connell J P A, Smith C W A, Reddy A S A, Rathmore K S. 2002. Cotton alpha-globulin promoter: isolation and functional characterization in transgenic cotton, Arabidopsis, and tobacco. Transgenic Research 11:347-359.
Suslow T V, Thomas B R, Bradford K J. 2002. Biotechnology provides new tools for planting. Oakland: University of California Division of Agriculture and Natural Resources, Publication 8043.
van der Geest A H M, Hall T C. 1996. A 68 bp element of the beta-phaseolin promoter functions as a seed-specific enhancer. Plant Mol. Biol. 32:579-588.
Vilardell J E, Martínez-Zapater J M E, Goday A E, Arenas C E, Pagès M. 1994. Regulation of the rab17 gene promoter in transgenic Arabidopsis wild-type, ABA-deficient and ABA-insensitive mutants. Plant Mol. Biol. 24:561-569.
Wilson W W, Jansen E L, Dahl B L. 2003. Issues in development and adoption of genetically modified (GM) wheat. AgBioForum 6:1-12.

All citations are hereby incorporated by reference.

The present invention has been described with regard to one or more embodiments. However, it will be apparent to persons skilled in the art that a number of variations and modifications can be made without departing from the scope of the invention as defined in the claims.

What is claimed is:

1. A method of identifying and tracing seeds, or fractions thereof, admixed with seeds from a different source using a seed colour marker that is dominant and is visually apparent in the hybrid seed.

2. The method of claim 1 wherein the seeds are two different transgenic seeds, one carrying one marker and the other carrying a second marker.

3. The method of claim 1 wherein the seeds are from a transgenic plant and the seeds from a different source are from a non-transgenic source.

4. The method of claim 1 wherein the seeds are from a novel-trait plants and the seeds from a different source are from a non-transgenic source.

5. The method of claim 1 wherein the fractions are selected from bran, endosperm, and germ from a classical milling method.

6. The method of claim 1 wherein the fractions are obtained from an air-classification milling method.

7. The method of claim 1 wherein the method includes introducing into a transgenic triticale cultivar a seed colour marker from wheat.

8. The method of claim 1 wherein the seed colour marker is produced by an anthocyanin gene.

9. The method of claim 8 wherein the seed colour marker is selected from red, blue, purple, grey, brown and black.

10. The method of claim 1 wherein the method includes introducing into a wheat or triticale cultivar Bperu and/or C1 regulatory genes under the control of a tissue-specific promoter whereby producing wheat or triticale plants with a distinct tissue colour.

11. The method of claim 1 wherein the method includes introducing into a wheat or triticale cultivar Bperu and/or C1 regulatory genes under the control of an embryo-specific promoter whereby producing wheat or triticale plants with a purple embryo.

12. The method of claim 1 wherein the method includes introducing into a wheat or triticale cultivar Bperu and/or C1 regulatory genes under the control of an endosperm-specific promoter whereby producing wheat or triticale plants with a purple embryo.

13. The method of claim 1 wherein the method includes introducing into a wheat or triticale cultivar Bperu and/or C1 regulatory genes under the control of a bran-specific promoter whereby producing wheat or triticale plants with a purple embryo.

14. The method of claim 1 wherein the method includes introducing into a wheat or triticale cultivar Bperu and/or C1 regulatory genes under the control of an aleurone-specific promoter whereby producing wheat or triticale plants with a purple embryo.

15. The method of claim 1 wherein the method includes introducing into a wheat or triticale cultivar Bperu and/or C1 regulatory genes under the control of an aleurone cell layer-specific promoter whereby producing wheat or triticale plants with a purple embryo.

16. The method of claim 11 wherein the embryo-specific promoter is selected from the group consisting of: a barley Ltp 1 promoter, a tobacco Itr1 promoter, a bean phas promoter, a cotton α-globulin promoter, a GS1-2 promoter, a barley Lox1 5′ promoter, a maze Adh1 promoter, a barley Dhn12 promoter, a a pea ps1 promoter, a maze rab17 promoter, a maze rab28 promoter and a tobacco napA promoter.

17. The method of claim 1 wherein the method includes producing a wheat cultivar with a black seed colour by crossing a wheat with a blue seed colour with a wheat with a purple seed colour.

18. A triticale plant produced by crossing a triticale plant with a wheat plant having a purple or blue seed colour, wherein said triticale plant so produced displays a seed coat colour selected from the group consisting of: blue, purple, red, grey, brown and black.

19. A wheat or triticale plant transformed with Bperu and/or C1 regulatory genes under the control of a tissue specific promoter whereby producing wheat or titicale plants with a distinct tissue colour.

20. The plant of claim 19 wherein the tissue specific promoter is selected from the group consisting of: an embryo-specific promoter, an endosperm-specific promoter, a bran-specific promoter, an aleurone-specific promoter and an aleurone cell layer-specific promoter.

21. The plant of claim 20 wherein the embryo-specific promoter is selected from the group consisting of: a barley Ltp 1 promoter, a tobacco Itr1 promoter, a bean phas promoter, a cotton α-globulin promoter, a GS1-2 promoter, a barley Lox1 5′ promoter, a maze Adh1 promoter, a barley Dhn12 promoter, a a pea psl promoter, a maze rab17 promoter, a maze rab28 promoter and a tobacco napA promoter.
